(19) United States
(12) Patent Application Publication
(10) Pub. No.: US 2006/0058388 A1
(43) Pub. Date: Mar. 16, 2006

(54) Methods for treating ocular diseases via introxide and or polyhydroxy acid containing compounds

(76) Inventor: Eric F Bernstein, Gladwyne, PA (US)

Correspondence Address: LICATLA & TYRRELL P.C., 66 E. MAIN STREET, MARLTON, NJ, 08053, US

(21) Appl. No.: 10/541,348

(22) PCT Filed: Dec. 31, 2003
(86) PCT No.: PCT/US03/41834

§ 371 Date: Sep. 26, 2005

Related U.S. Application Data
(60) Provisional application No. 60/438,267, filed on Jan. 3, 2003.

(51) Int. Cl. A61K 31/21; A61K 31/19

(52) U.S. Cl. 514/557; 514/509

(57) ABSTRACT

Methods for prevention or treatment of ocular diseases via administration of a nitroxide and/or polyhydroxy acid containing compound are provided.

### FIELD OF THE INVENTION

The present invention relates to new uses for compositions comprising a nitroxide and/or polyhydroxy acid containing compounds for treatment of ocular disease such as macular degeneration, cataracts and glaucoma.

### BACKGROUND OF THE INVENTION

Nitroxides are stable free radicals with antioxidant catalytic activities similar to superoxide dismutase. Nitroxides existing in vivo have been shown to interact with other substances to also mimic catalase activities. Thus, nitroxide containing compounds have been described in the art for numerous uses. For example, U.S. Pat. No. 5,462,946 discloses biologically compatible compositions containing an effective amount of a metal independent nitroxide compound for use in protecting the skin against ionizing radiation, mucositis, the effects of whole body radiation and radiation induced hair loss. In this embodiment, the nitroxide containing composition is applied topically as an ointment, lotion or cream, intravenously or orally by pill or lozenge. This patent also teaches the nitroxide containing compounds to be useful as protectants against: increased oxygen exposure so as to avoid pulmonary adult respiratory distress syndrome; oxygen-induced lenticular degeneration and hyaline membrane disease in infants; oxidative stress-induced cataracts; reperfusion injury in treating cardiovascular phenomena such as myocardial infarction and strokes, pancreatitis or intestinal ulceration and organ transplant; cytotoxicity due to excess oxidation in animal or plant cell cultures; cytotoxic effects of chemotherapeutic agents; and mutagenic and carcinogenic agents. Also taught in this patent is use of these compounds as anti-inflammatory agents effective against arthritic conditions. In this embodiment, the nitroxide containing compositions are administered parenterally, intra-articularly or via oral ingestion. This patent also teaches use of these compounds as aging retardants when administered orally or parenterally and in weight reduction when administered orally or intravenously.

U.S. Pat. Nos. 5,824,781, 5,840,701 and 5,817,632 teach compositions and processes to alleviate free radical toxicity based on use of nitroxides in association with physiologically compatible macromolecules. These compositions are suggested to be useful as blood substitutes, radioprotective agents, imaging agents, agents to protect against ischemia and reperfusion injury, particularly cerebral stroke, and in vivo enzyme mimics.

Benzo-fused heterocyclic polyhydroxy acid containing compounds for use in treating dermatological, rheumatic, respiratory, cardiovascular and corneopathies are taught in U.S. Pat. No. 5,849,798 and U.S. Pat. No. 6,194,450. Phenyl benzoate derivatives with polyhydroxy acid substitutions for use in medicines and cosmetics for treating dermatological conditions relating to keratinization disorders, as antiinflammatories and as anti-immuno-allergic agents are taught in U.S. Pat. No. 5,332,856 and U.S. Pat. No. 5,468,897.

Methods and compositions containing hydroxyacids or related compounds for enhancing the therapeutic effects of cosmetic and pharmaceutical agents used in topical treatment of cosmetic conditions, dermatological disorders and other afflictions are described in U.S. Pat. No. 6,051,609 and U.S. Pat. No. 5,962,526.

### SUMMARY OF THE INVENTION

An object of the present invention is to provide pharmaceutical compositions for prevention and/or treatment of ocular diseases such as macular degeneration, cataracts and glaucoma comprising a nitroxide and/or polyhydroxy acid containing compound and a pharmaceutically acceptable vehicle.

Another object of the present invention is to provide methods for prevention and/or treatment of ocular diseases such as macular degeneration, cataracts, and glaucoma via administration of a nitroxide and/or polyhydroxy acid containing compound.

### DETAILED DESCRIPTION OF THE INVENTION

Macular degeneration is a disease affecting the macula, a thin area in the center of the retina that confers the ability for acute and detailed vision. The central area of the macula is known as the fovea and is composed entirely of cones. These cones have specialized structure that aids in detection of detail in a visual image. In this region, the blood vessels, ganglion cells, and plexiform layers are all displaced to one side to allow light to pass unimpeded to the cones.

Degeneration of the tissue in this area of the retina is primary cause of debilitating visual impairment in the elderly and a major cause of blindness in developing countries throughout the world. Two forms of the disease have been identified, geographic atrophy and choroidal neovascularization. This disease is most often linked to aging. However, macular degeneration can also result from hypertension in younger individuals.

Treatments currently used in patients with the choroidal neovascularization form of macular degeneration include photodynamic therapy, pharmacologic inhibition of choroidal neovascular membrane formation with anti-angiogenic agents, surgical intervention, including excision of subfoveal choroidal neovascular membranes, and radiation therapy (Ciulla et al. Surv. Opthalmol. 1998 43:134-146). The goal of therapy in each method of treatment is to destroy the choroidal neovascular membranes.

A cataract is a lens opacity and may be congenital, traumatic, or secondary to a systemic disease such as diabetes, myotonic dystrophy, or atopic dermatitis, systemic corticosteroid treatment, or uveitis. Senile cataract is the most common type withe most persons over the age of 60 having some degree of lens opacity. Primary treatment is surgical removal of the cataract.

Glaucoma is a disease of the eye characterized by increased intraocular pressure due to restricted outflow of the aqueous humor through the aqueous veins and Schlemm's canal, excavation and degeneration of the optic disk, and nerve fiber bundle damage producing arcuate defects in the field of vision. Treatments include topical administration of pharmacological agents such as β-adrenergic blocking agents such as timolol, levobunolol and metipranolol, β₁-receptor selective blocking agents such as betaxolol, epinephrine, α₂-agonists such as apraclonide, pilocarpine, prostaglandin analogues such as latanoprost, carbonic anhydrase inhibitors such as dorzolamine and acetazolamide. Additional treatments include laser trabeculoplasty as well as surgical trabeculectomy.

It is now believed that application of a composition comprising a nitroxide and/or polyhydroxy acid containing compound will be useful in treating ocular diseases such as macular degeneration, cataracts, and glaucoma. For purposes of the present invention, by “nitroxide” or “nitroxide containing compound” it is meant stable nitroxide free radicals. Examples of nitroxide containing compounds are well known in the art and taught in prior art references such as U.S. Pat. No. 5,462,946.

For purposes of the present invention, by “polyhydroxy acid” or “polyhydroxy acid containing compound” it is meant a compound, preferably an aromatic compound, with a polyhydroxy acid substitution. Exemplary polyhydroxy acid containing compounds are well known in the art and taught in prior art references such as U.S. Pat. No. 6,051,609 and U.S. Pat. No. 5,962,526.

Phototoxicity and cytotoxicity as well as the the ability of nitroxide and/or polyhydroxy acid containing compounds to protect again ultraviolet-induced gene induction were examined in human skin fibroblasts containing the human elastin promoter linked to a luciferase reporter gene construct. Glucolactone did not demonstrate significant phototoxicity or cytotoxicity, with the ED₅₀ for phototoxicity being approximately 102 g/L. Further glucolactone protected against UV-induced promoter induction approximately 50%. Thus, gluconolactone protects against UV-induced gene induction in cells containing the human elastin promoter/luciferase construct. Tempol also demonstrated protection against increasing amounts of ultraviolet radiation in the neutral red assay. The ED₅₀ for cell cultures with tempol but not receiving any ultraviolet radiaiton was approximately 100 mM. The ED₅₀ for cell cultures with tempol and receiving ultraviolet radiation doses of 2 and 5 mJ/cm² was approximately 150 and 250 mM, respectively. Further, at increasing ultraviolet doses tempol protected significantly against toxicity in both the neutral red assay and the human elastin promoter/luciferase assays, demonstrating over 50% protection.

Accordingly, these experiments are indicative of nitroxide and/or polyhydroxy acid containing compounds providing a safe means for protecting against toxicities of ultraviolet radiation in cells and providing useful treatment for oculat disease such as macular degeneration, cataracts and/or glaucoma.

Nitroxide and/or polyhydroxy acid containing compounds can be formulated with a pharmaceutically acceptable vehicle selected in accordance with the route of administration. For example, for production of a pharmaceutical composition which can be applied directly to the eye, the compositions may be formulated either as a cream or in solution; for oral administration, the compositions may be formulated as a tablet, capsule or in solution; for intravenous administratation or adminstration via injection parenterally or intramsucularly, the compound is prefereably dissolved in saline or phosphate buffered saline, etc. Formulations of such pharmaceutical compositions can be produced routinely by one of skill in the art in accordance with well known techniques. In one embodiment of the present invention, the pharmaceutical compositions comprises either a nitroxide containing compound or a polyhydroxy acid containing compound. More preferred are pharmaceutical compositions comprising both a nitroxide containing compound and a polyhydroxy acid containing compound.

These pharmaceutical compositions can be administered to a patient suffering from an ocular disease such as macular degeneration, cataracts, and glaucoma for treatment thereof. These pharmaceutical compositions can also be adminsitered to a patient to prevent or slow development of ocular diseases such as macular degeneration, cataracts, and glaucoma. In a preferred embodiment, the compositions is administered directly to the eye. However, the composition can also be administered orally, intravenously, sublingually, intramuscularly, via suppository, or any other route wherein the compounds maintain their ability to treat the ocular disease. For purposes of the present invention, by treatment, treat to treating, it is meant an alleviation or decrease in the symptoms of a patient suffering from macular degeneration.

The following nonlimiting examples are provided to further illustrate the present invention.

### EXAMPLES

### Example 1

### Fibroblast Cultures

Immortalized human skin fibroblasts containing the human elastin promoter linked to a luciferase reporter gene construct were seeded in 96-well plates at a density of 7,500 cells per well (doubling time ˜30 hours). Cells were grown in 75 cm² tissue culture flasks in MEM (without phenol red) supplemented with 10% FBS, 25 mM HEPES, 2 mM l-glutamine, 1% nonessential amino acids, 50 μg/mL Gentamycin, and 5 μg/mL Zeocin. Cell cultures were incubated in a 37° C. incubator (containing 5% CO₂) when not being manipulated or irradiated. Cell cultures were washed with Phosphate Buffered Saline (PBS) with Ca⁺⁺ and Mg⁺⁺ when media or compounds were removed and before ultraviolet radiation (UVR).

### Example 2

### Gluconolactone and Tempol

Gluconolactone was prepared in PBS. Concentrations of 0.5, 1.0, 2.5, 5.0, 7.5, 10.0, 12.5, and 25% (w/v) were prepared and adjusted to a pH of 7.2. Gluconolactone was incubated 15 minutes prior to and in contact with cell cultures during UV-irradiation.

4-Hydroxy-tempo (tempol) was prepared in PBS. Concentrations of tempol of 10, 25, 50, 62.5, 100, 125, 250, and 500 mM were prepared and adjusted to a pH of 7.2. Tempol was incubated 15 minutes prior to and in contact with cell cultures during UV-irradiation.

### Example 3

### Ultraviolet Radiation

Cell cultures were UV-irradiated with Westinghouse 40-Watt fluorescent sunlamps (FS-40 lamps) with doses of either 2 or 5 mJ/cm², based on a PMA2100 radiometer (Solar light Company, Philadelphia, Pa.) equipped with a PMA2101 erythema weighted detector.

### Example 4

### Neutral Red Cytotoxicity/Phototoxicity Assay

Fifteen hours after UV-irradiation with the FS-40 lamps, cell cultures were washed with PBS and Neutral Red Medium (Neutral Red dye+supplemented MEM) was added for a 3 hours (in 37° C. incubators). After the incubation period, cells are washed with PBS and the Neutral Red Desorb solution (50% Ethanol, 49% distilled water, 1% Glacial Acetic Acid) was added. Plates are shaken for 10 minutes at 500 rpm and then the absorption was read at 540 nm using THERMOmax plate reader and SOFTmax PRO software.

What is claimed is:

1. A method for treatment of ocular diseases comprising administering to a patient suffering from an ocular disease a nitroxide containing compounds or polyhydroxy acid containing compounds.

2. The method of claim 1 wherein the ocular disease is macular degeneration, cataracts, or glaucoma.

3. A method for treatment of ocular diseases comprising administering to a patient suffering from an ocular disease a nitroxide containing compound and a polyhydroxy acid containing compound.

4. The method of claim 3 wherein the ocular disease is macular degeneration, cataracts, or glaucoma.

5. A method for preventing ocular diseases comprising administering to a subject a nitroxide containing compounds or polyhydroxy acid containing compounds.

6. The method of claim 5 wherein the ocular disease is macular degeneration, cataracts, or glaucoma.

7. A method for preventing ocular diseases comprising administering to a subject a nitroxide containing compound and a polyhydroxy acid containing compound.

8. The method of claim 7 wherein the ocular disease is macular degeneration, cataracts, or glaucoma.

9. A pharmaceutical composition for treatment or prevention of ocular diseases comprising a nitroxide containing compound, a polyhydroxy acid containing compound, and a pharmaceutically acceptable vehicle.

10. The pharmaceutical composition of claim 9 wherein the ocular disease is macular degeneration, cataracts, or glaucoma.
